(19) United States
(12) Patent Application Publication
(10) Pub. No.: US 2025/0344672 A1
(43) Pub. Date: Nov. 13, 2025

(54) LIGHT THERAPY BLANKET

(71) Applicant: EQUISOL LLC, Folsom, LA (US)

(72) Inventor: Jackie JOLIE, Bush, LA (US)

(73) Assignee: EQUISOL LLC, Folsom, LA (US)

(21) Appl. No.: 18/867,391

(22) PCT Filed: Jul. 17, 2023
(86) PCT No.: PCT/US23/27941

§ 371 Date: Nov. 19, 2024

Related U.S. Application Data
(60) Provisional application No. 63/390,135, filed on Jul. 18, 2022.

(51) Int. Cl. A01K 13/00; A61N 5/06

(52) U.S. Cl. CPC A01K 13/008; A61N 5/0613; A61N 2005/0645; A61N 2005/0652; A61N 2005/0659; A61N 2005/0663

(57) ABSTRACT

This invention provides an animal therapy blanket. The inventive blanket provides whole-body inflammation therapy. It comprises a fabric that is shaped to mold to the body of a given animal and is secured with adjustable straps. The inner side of the blanket comprises a plurality of openings through which light emitting diodes are passed through to contact the skin of the animal. The diodes comprise LED lights at infrared and red frequencies designed to promote healing within the animal.

### CROSS-REFERENCE TO RELATED APPLICATIONS

This application claims the benefit of U.S. Provisional Application No. 63/390,135 entitled “Light Therapy Blanket” and filed Jul. 18, 2022.

### STATEMENT REGARDING FEDERALLY SPONSORED RESEARCH OR DEVELOPMENT

Not Applicable

### BACKGROUND OF THE INVENTION

### Field of the Invention

This invention relates to the field of animal rehabilitation, especially through light therapy.

### Background Information

Like their human counterparts, our four-legged pets live indoors more, subjecting them to more toxins and environmental stress and causing them to become weaker, inflamed, and at more risk for injuries and chronic disease. Being kept indoors or stabled for most of the day causes the body to get out of sync with the biological rhythms of the natural light spectrum available outside with the sun. Every mammal has natural biological clocks in their bodies and that system, called circadian rhythms, keep each bodily system synced and functioning together. When the circadian rhythms of the body are disrupted by living indoors more; chronic inflammation sets in, which has been proven to lead to hormonal imbalances, autoimmune diseases, chronic pain, injuries, and even death.

In the last two to three decades, horses and dogs have succumbed to more injuries, autoimmune issues, cancers, and other diseases that didn't exist when they were living outside and in sync with the natural spectrum of light. In our modern world and especially, for example, the competitive world of horses and dogs, it is typically not convenient or possible to house these animals outside and in the sun.

Light therapy has been known for a long time, and over 100,000 scientific studies show the health and performance benefits of red and infrared wavelength light therapy. Health benefits of using red/infrared light therapy include: Reduce inflammation in cells; increase blood circulation to the tissue; improve wound healing and improve scars; increase collagen production which protects soft tissue; increases energy (ATP) in the cells; increases lymphatic system activity (detox and decrease swelling); helps relax muscles; stimulates trigger and acupuncture points; helps release endorphins to help with pain relief; stimulates Nitric Oxide production, which can help in peripheral neuropathies; increases RNA and DNA synthesis to help repair damaged cells; help in bone healing (fractures, breaks).

In the animal industry, light therapy is used either by a concentrated cold red-light laser or red/infrared light therapy pads to use in localized areas. This options only address localized and specific areas of inflammation. However, animals' bodies are under constant environmental stress and have generalized inflammation throughout the entire body when their circadian rhythms are dysfunctional.

The current invention solves this issue by providing a red and infrared light therapy blanket for animals, such as horses and dogs. This invention is an innovative and therapeutic solution to relieve an animal's body of systemic inflammation. In addition to decreasing whole body inflammation, the invention also aids in many other rehabilitation and performance factors for the animals. Using this inventive light therapy blanket brings a whole-body approach to healing and enhancing performance that is currently not being provided in the animal health industry.

### SUMMARY OF THE INVENTION

This invention is aimed at solving the persuasive problem in the animal therapy industry of a lack of whole-body inflammation therapies. In one embodiment, the invention comprises a novel blanket that is shaped to mold to the body of a given animal and is secured with adjustable straps. The inner side of the blanket comprises a plurality of openings through which light emitting diodes are passed through to contact the skin of the animal. The diodes comprise LED lights at infrared and red frequencies designed to promote healing within the animal.

Whether an animal lives a more athletic or recreational lifestyle, the inventive blanket provides therapeutic benefits. Use of the blanket reduces inflammation in the body, calms the animal, and relaxes the muscles. The blanket may also be used as a warming aid. The blanket also assists in rehabilitation cases such as colic, kissing spine, ACL tears, and other surgeries by preventing scar tissue and hydrating the surrounding tissues to heal faster. Infrared light has been studied and proven to increase cellular ATP (energy) and hydrate soft tissue allowing the body to move and perform better.

### BRIEF DESCRIPTION OF THE DRAWINGS

The drawings constitute a part of this specification and include exemplary embodiments of the Light Therapy Blanket, which may be embodied in various forms. It is to be understood that in some instances, various aspects of the invention may be shown exaggerated or enlarged to facilitate an understanding of the invention. Therefore, the drawings may not be to scale

FIG. 1 is an embodiment of the invention for equine therapy.

FIG. 2 is an embodiment of the invention for equine therapy shown secured to an animal.

FIG. 3 is an embodiment of the invention for equine therapy shown secured to an animal depicting the LED diodes actuated.

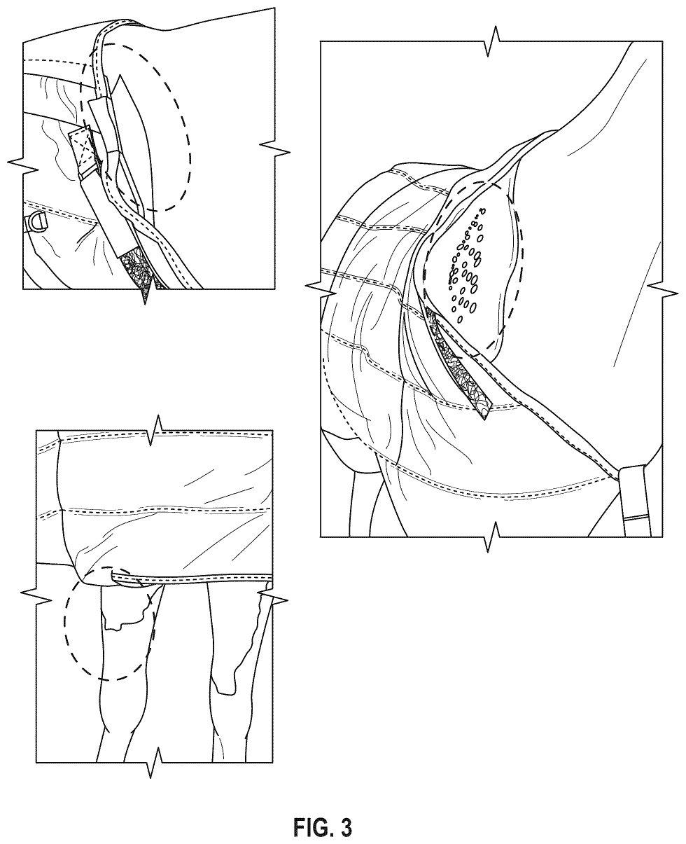

FIG. 4 is an embodiment of the invention for small animal therapy.

FIG. 5 is an embodiment of the invention for small animal therapy shown secured to an animal.

FIG. 6 is an embodiment of the invention for small animal therapy shown secured to an animal depicting the LED diodes actuated.

### DETAILED DESCRIPTION OF THE INVENTION

The subject matter of the present invention is described with specificity herein to meet statutory requirements. However, the description itself is not intended to necessarily limit the scope of claims. Rather, the claimed subject matter might be embodied in other ways to include different steps or combinations of steps similar to the ones described in this document, in conjunction with other present or future technologies.

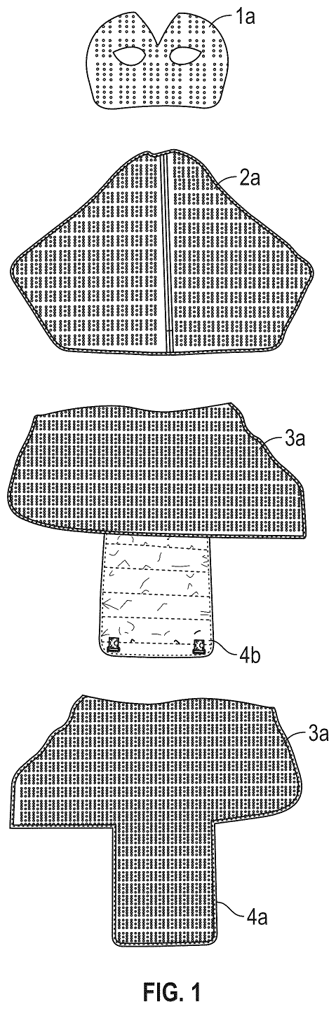

FIG. 1 shows one embodiment of the invention that comprises an equine blanket.

In one or more embodiments, the outer surface of the blanket 1b, 2b, 3b, 4b comprises nylon in a suitable color, for instance black. Other suitable materials are contemplated such as wool, rayon, polyester, bamboo, and cotton. The outer surface of the blanket comprises a face portion 1b, a neck piece 2b, a body portion 3b, and a fastening portion 4b. The inner surface of the blanket, 1a, 2a, 3a, 4a, the side against the body of the animal when in use, comprises neoprene padding. The neoprene padding provides protective and insulating qualities to the inner LED lights, discussed below. Any suitable material that meets these requirements is contemplated, such as Thermocline® or silicone rubber.

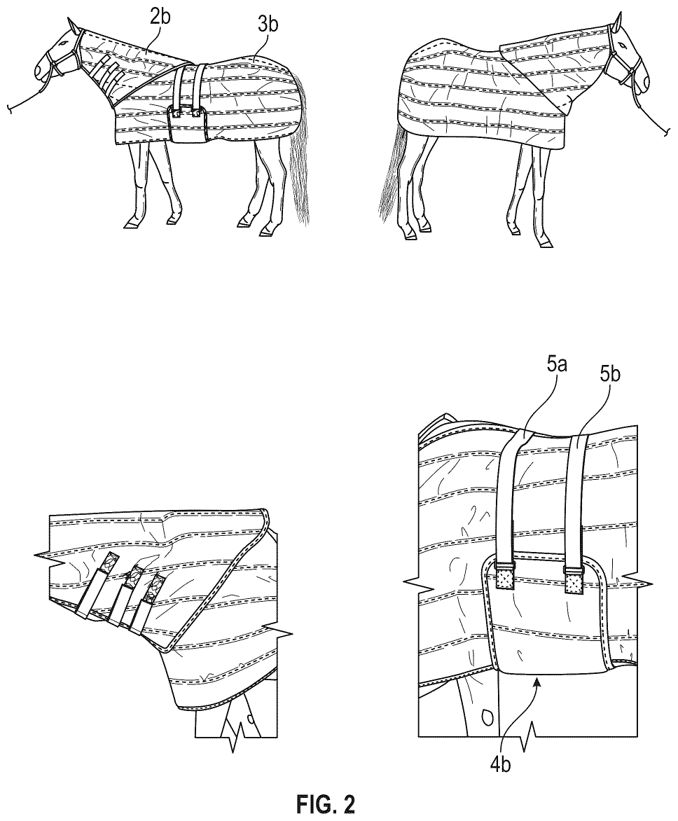

As shown in FIG. 1, the face portion 1a, 1b is designed to rest on the animal's face. It provides therapeutic benefits not just on the torso of the animal, but also provides benefits to the animal's face muscles, tendons, etc., such as the TMJ joint. The neck piece 2a, 2b is designed for equine animals, but can be used in any embodiment. The neck piece is designed to provide therapeutic benefits along the length of the neck of the animal and is designed to follow the contours of the neck. An embodiment of the neck piece 2a, 2b is shown in FIG. 2 at element 2. In one or more embodiments, other pieces are contemplated, such as a boot shaped piece for the animal's legs.

The body portion 3a, 3b is designed to match the contours of the torso and rump of the animal. The body portion 3a, 3b is secured with the fastening portion or flap 4a, 4b. In one embodiment, the fastening portion or flap 4a, 4b fits around the stomach of the animal and is secured using straps 5a, 5b. In the depicted embodiment, the straps 5a, 5b fit around the torso area of the animal and are connected to the fastening portion or flap 4a, 4b such that the fastening portion or flap 4a, 4b fits snuggly against the underside or stomach area of the animal. This is shown in FIG. 2. The straps 5a, 5b are secured using a loop and may be adjusted to ensure a secure fit. In other embodiments, other known adjustable fastening means may be used such as Velcro or snaps. The neck piece 2a, 2b is also secured using a similar adjustable means.

The blanket may be sized to accommodate differing sizes of the animal. For example, the blanket may be small (72 inches in length), medium (78 inches in length), or large (84 inches in length). The blanket is preferably fitted so that it fits right above the knee of the animal, as shown in FIG. 2.

The inner surface of the blanket, 1a, 2a, 3a, 4a comprises a plurality of LED lights as shown at 1a, 2a, 3a, 4a in FIG. 1. LED light therapy promotes healing of the body. The energy (photons) from the lights increases the energy within the cell, which accelerates the healing process. The lights must be on specific wavelengths and must be delivered at a specific dosage.

In a preferred embodiment, the LED lights comprises LED diodes that protrude through the inner surface of the blanket 1a, 2a, 3a, 4a in such a way as to contact the skin of the animal. Accordingly, the inner surface of the blanket 1a, 2a, 3a, 4a comprise a plurality of openings through which to fit the diodes.

In one embodiment, both red and near infrared lights are used because the wavelengths are absorbed by different depths of tissue. The combination of the two works in concert to provide benefits for soft tissue injuries, inflammation, ligament soreness, tendon problems, sore backs, splints, strains, stifle issues, sprains, swelling, shoulder pain, hip pain, sore backs, sore necks, salivary gland problems, wounds, cuts, scrapes, arthritis pain and for trigger points and acupuncture points. Visible red light (660 nm) is absorbed by skin layers very efficiently and is best for uses such as stimulating trigger and acupuncture points and for treating wounds and infections. Near infrared light (850 nm) penetrates to a deeper level and has been used to treat concerns of tendons, ligaments, bones, joints and muscle.

In one embodiment, the LED lights are at alternating 660 nm (red LED) and 850 nm (far infrared) wavelengths. However, other wavelengths are contemplated.

In order to power the lights, a rechargeable battery pack may be used. The battery pack is located within the inner surface and outer surface of the blanket. Other power options are contemplated such as replaceable battery packs and solar panels or solar chargeable batteries. A timer may also be used to time out the therapy. In a preferred embodiment, the timer automatically shuts off the LED lights after 15-minute sessions. Other timer options are contemplated, including that the timer may be adjusted to session times based on the amount of light emitted, the number of LED lights, or the size of the blanket and animal.

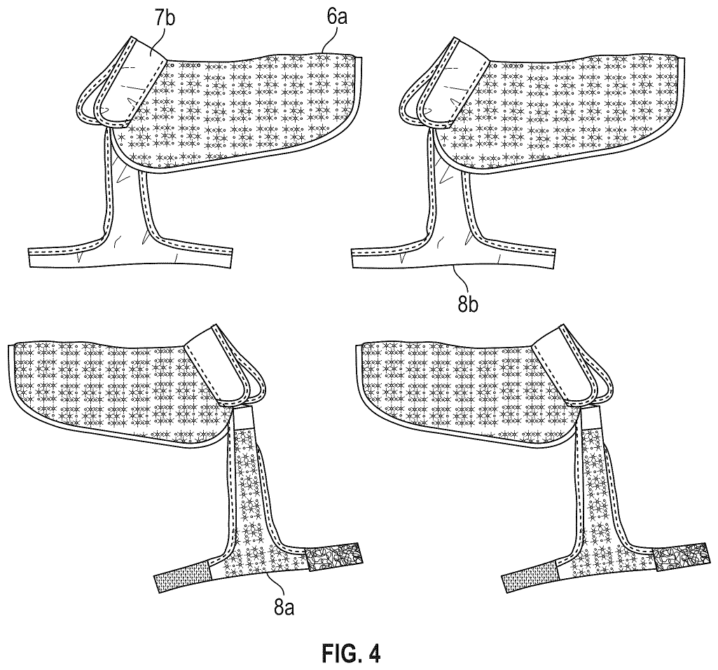

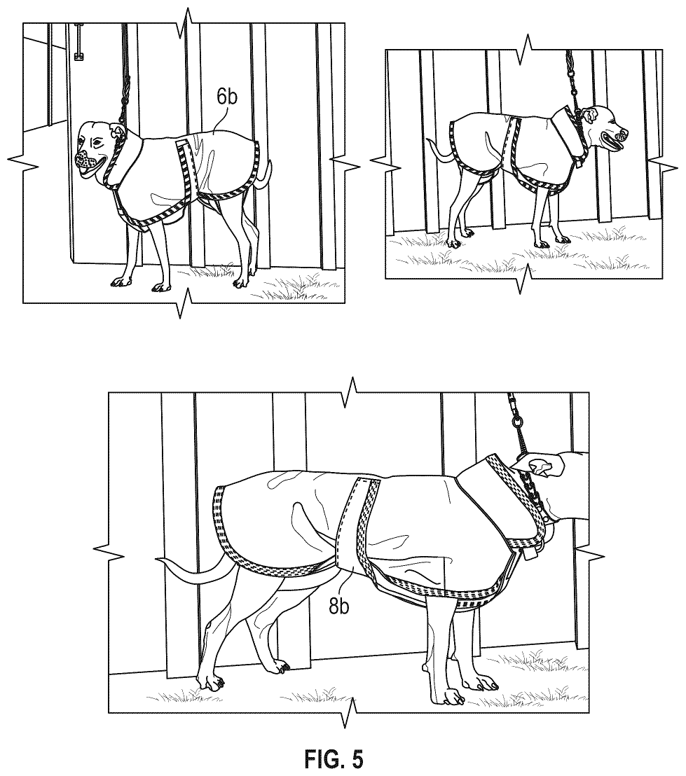

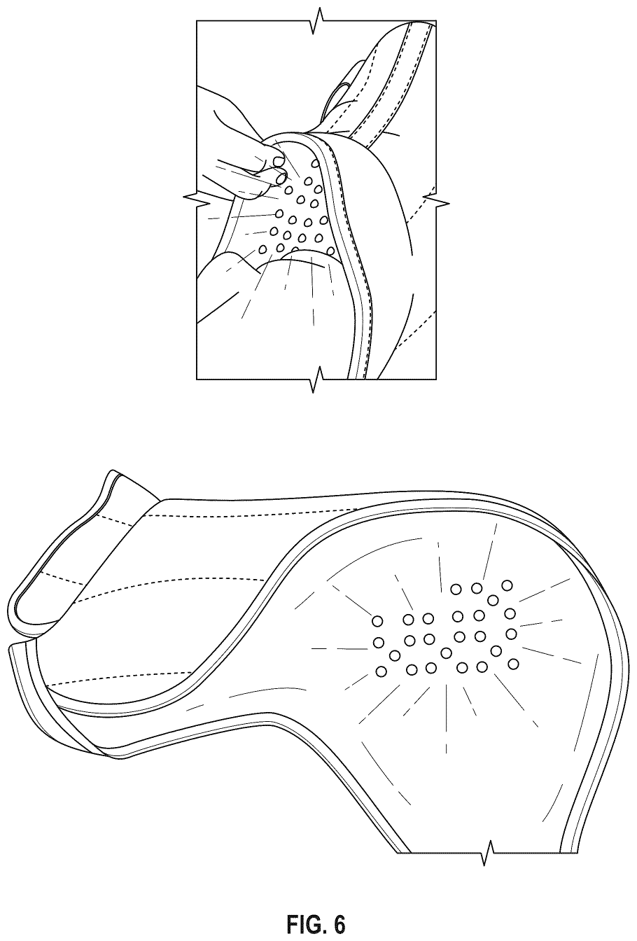

Another embodiment of the inventive blanket is show in FIGS. 4, 5 and 6. In this embodiment, the blanket is smaller to fit other four-legged animals such as cats, dogs, goats and other domestic animals as well as large cats, llamas, foxes, bore, etc., which could be used by wildlife sanctuaries or zoos.

In this embodiment, there is a similar body portion 6a, 6b that is designed to fit a smaller animal's torso and rump. The inner surface and outer surface are made of similar materials discussed in the previous embodiment. As shown in FIGS. 4, 5 and 6, the body portion 6a, 6b may also comprise a neck cuff 7b so provide therapy to the animal's neck and shoulder area. This embodiment also comprises a fastening portion 8a, 8b that uses straps to wrap around the animal to secure the blanket in place. In one embodiment, the fastening portion 8a, 8b is connected at the front of the body piece, near the animal's head and is wide enough to fit the chest and stomach area of the animal so that when the strap passes over the animal's chest to the stomach area where it is wrapped around the animal, those areas receive treatment as well.

LED lights 9 discussed in the embodiment above are also applied to this embodiment to provide treatment, as best shown in FIG. 9.

In order to use the blanket, ensure that the battery is charged, fit the blanket around the animal and adjust the straps so that it is secure. Set the timer for the desired treatment time. Remove the blanket after treatment. Repeat as necessary. The blanket may be used with all pieces, or with some subset.

A preferred embodiment of the invention is as follows: an inner surface comprising a plurality of openings and an outer surface and a torso edge and a rump edge wherein said blanket extends laterally from said torso edge to said rump edge such that the blanket comprises a first side edge and a second side edge, and said outer surface comprises and interior and exterior side and wherein a plurality of LED diodes are dispersed on said interior side of said outer surface and protrude through said inner surface through said plurality of openings; and a flap that extends from said first side edge the midline of said first side edge, said flap having a longitudinal length that is less than the longitudinal length of said first side edge and said flap comprising at least one strap and said at least one strap consists of a flap side and a body side, and wherein said outer surface comprises a connector that accepts said body side of said at least one strap so that said at least one strap can be removably attached to said body side. The blanket may also comprise a face portion, wherein said face portion comprises an inner surface comprising a plurality of openings and an outer surface comprising a plurality of LED diodes wherein said LED diodes protrude through said inner surface.

Another preferred embodiment of the invention is as follows: the blanket comprises a body portion and a neck portion. Said body portion comprising: an outer surface and an inner surface and wherein a plurality of LED diodes are dispersed on said body portion outer surface and protrude through said body portion inner surface; a left side and a right side such that when said body portion is folded at its centerline, said left side corresponds to the left side of the torso of said animal and said right side corresponds to the right side of the torso of said animal; and at least one strap that extends from said right side and is removably attached to said left side. Said neck portion comprises an outer surface and an inner surface and wherein a plurality of LED diodes are dispersed on said neck portion outer surface and protrude through said neck portion inner surface; a left side and a right side such that when said neck portion is folded at its centerline, said left side corresponds to the left side of the face and neck of said animal and said right side corresponds to the right side of the face and neck of said animal; and at least one neck portion strap that extends from said right side and is removably attached to said left side. The neck portion strap and/or body portion strap may instead extend from said left side and is removably attached to said right side.

A third preferred embodiment of the invention is as follows: a body portion and an integrated neck cuff, said body portion and said integrated neck cuff comprising: an inner surface comprising a plurality of openings and an outer surface, and said outer surface comprises and interior and exterior side and wherein a plurality of LED diodes are dispersed on said interior side of said outer surface and protrude through said inner surface through said plurality of openings; and a flap that extends from said body portion at said integrated neck cuff. In this embodiment, aid flap further comprises a strap.

For the purpose of understanding the Light Therapy Blanket, references are made in the text to exemplary embodiments of a Light Therapy Blanket, only some of which are described herein. It should be understood that no limitations on the scope of the invention are intended by describing these exemplary embodiments. One of ordinary skill in the art will readily appreciate that alternate but functionally equivalent components, materials, designs, and equipment may be used. The inclusion of additional elements may be deemed readily apparent and obvious to one of ordinary skill in the art. Specific elements disclosed herein are not to be interpreted as limiting, but rather as a basis for the claims and as a representative basis for teaching one of ordinary skill in the art to employ the present invention.

1. A light therapy animal blanket comprising:
a. an inner surface comprising a plurality of openings and an outer surface and a torso edge and a rump edge wherein said blanket extends laterally from said torso edge to said rump edge such that the blanket comprises a first side edge and a second side edge, and said outer surface comprises and interior and exterior side and wherein a plurality of LED diodes are dispersed on said interior side of said outer surface and protrude through said inner surface through said plurality of openings; and
b. a flap that extends from said first side edge the midline of said first side edge, said flap having a longitudinal length that is less than the longitudinal length of said first side edge and said flap comprising at least one strap and said at least one strap consists of a flap side and a body side, and wherein said outer surface comprises a connector that accepts said body side of said at least one strap so that said at least one strap can be removably attached to said body side.

2. The light therapy animal blanket of claim 1 further comprising a face portion, wherein said face portion comprises an inner surface comprising a plurality of openings and an outer surface comprising a plurality of LED diodes wherein said LED diodes protrude through said inner surface.

3. A light therapy animal blanket comprising:
a. a body portion, said body portion comprising:
i. an outer surface and an inner surface and wherein a plurality of LED diodes are dispersed on said body portion outer surface and protrude through said body portion inner surface;
ii. a left side and a right side such that when said body portion is folded at its centerline, said left side corresponds to the left side of the torso of said animal and said right side corresponds to the right side of the torso of said animal; and
iii. at least one strap that extends from said right side and is removably attached to said left side.
b. a neck portion, said neck portion comprising:
i. an outer surface and an inner surface and wherein a plurality of LED diodes are dispersed on said neck portion outer surface and protrude through said neck portion inner surface;
ii. a left side and a right side such that when said neck portion is folded at its centerline, said left side corresponds to the left side of the face and neck of said animal and said right side corresponds to the right side of the face and neck of said animal; and
iii. at least one neck portion strap that extends from said right side and is removably attached to said left side.

4. The light therapy animal blanket of claim 3 wherein said neck portion strap instead extends from said left side and is removably attached to said right side.

5. The light therapy animal blanket of claim 3 wherein said body portion strap instead extends from said left side and is removably attached to said right side.

6. A light therapy animal blanket comprising:
a. a body portion and an integrated neck cuff, said body portion and said integrated neck cuff comprising: an inner surface comprising a plurality of openings and an outer surface, and said outer surface comprises and interior and exterior side and wherein a plurality of LED diodes are dispersed on said interior side of said outer surface and protrude through said inner surface through said plurality of openings; and
b. a flap that extends from said body portion at said integrated neck cuff.

7. The light therapy animal blanket of claim 5 wherein said flap further comprises a strap.
